(19) United States
(12) Patent Application Publication
(10) Pub. No.: US 2023/0345936 A1
(43) Pub. Date: Nov. 2, 2023

(54) NOVEL ENVIRONMENT-FRIENDLY HERBICIDE

(71) Applicants: KUNMING INSTITUTE OF BOTANY, CHINESE ACADEMY OF SCIENCES, Kunming City (CN); YUNNAN AGRICULTURAL UNIVERSITY, Kunming City (CN)

(72) Inventors: Zhiyun YANG, Kunming City (CN); Xiao DING, Kunming City (CN); Junbo YANG, Kunming City (CN); Guoxing WU, Kunming City (CN); Mingxian LAN, Kunming City (CN)

(21) Appl. No.: 17/900,091

(22) Filed: Aug. 31, 2022

(30) Foreign Application Priority Data
Apr. 28, 2022 (CN) 202210470649.4

(51) Int. Cl. A01N 37/02; A01N 25/04

(52) U.S. Cl. CPC A01N 37/02; A01N 25/04

(57) ABSTRACT

The present disclosure provides a use of a novel environment-friendly agricultural chemical, and belongs to the field of agricultural chemicals. It is found in the present disclosure that methyl 4-hydroxyphenylacetate may be used for controlling plants and preparing herbicides. The methyl 4-hydroxyphenylacetate in the present disclosure has a use concentration greater than 150 mmol/L. The herbicide is prepared by using the methyl 4-hydroxyphenylacetate as an active ingredient in the present disclosure has a good controlling and killing effect on perennial herbs or Half-shrubby plants, especially Eupatorium adenophorum, and has low toxicity, no harm, and no solvent residues after use.

### CROSS REFERENCE TO RELATED APPLICATION

This patent application claims the benefit and priority of Chinese Patent Application No. 202210470649.4, entitled use of novel environment-friendly agricultural chemical filed on Apr. 28, 2022, the disclosure of which is incorporated by reference herein in its entirety as part of the present application.

### TECHNICAL FIELD

The present disclosure belongs to the field of agricultural chemicals, and in particular, to a novel environment-friendly herbicide.

### BACKGROUND ART

Herbicides are agents that can kill weeds completely or selectively, also known as weedicides, and are a class of substances used to eliminate weeds or inhibit plant growth. At present, paraquat and nitrofen are the main chemical herbicides. Paraquat is the most widely used room temperature herbicide in China. After spraying, the paraquat causes toxic residues, damages the environment, is extremely toxic to humans, and has no specific antidote. From Sep. 26, 2020, paraquat sol has been banned from sales and use in China. Nitrofen is a contact herbicide used at pre-emergence or early post-emergence, which can kill most of the Annual weedannual weeds and has a certain inhibitory effect on some perennial weeds, but the weeds cannot be eradicated. The nitrofen has certain toxicity, and is also a agricultural chemical banned from production, sales and use.

Methyl 4-hydroxyphenylacetate, an important pharmaceutical intermediate, is cream-colored crystalline powder with a chemical formula of C₉H₁₀O₃, and has a molecular weight of 166.1739, a density of 1.181 g/cm³, a melting point of 55-58° C., a boiling point of 162-163° C., a flash point of 163° C., and a structure as follows:

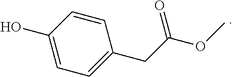

At present, there is no report on application methods of the methyl 4-hydroxyphenylacetate in herbicides.

### SUMMARY

In view of this, an objective of the present disclosure is to provide a method for using methyl 4-hydroxyphenylacetate in control of plants. The plants are perennial herbs or Half-shrubby plants, and the methyl 4-hydroxyphenylacetate especially has an excellent contact killing effect on Eupatorium adenophorum.

To achieve the above objective, the present disclosure provides the following technical solutions.

The present disclosure provides a method for using methyl 4-hydroxyphenylacetate in control of a plant.

Preferably, the plant may include perennial herbs or Half-shrubby plants.

Preferably, the plant may be Eupatorium adenophorum.

Preferably, the methyl 4-hydroxyphenylacetate may have a use concentration greater than 150 mmol/L.

The present disclosure further provides a method for using methyl 4-hydroxyphenylacetate in herbicides.

The present disclosure further provides a herbicide. Active ingredients of the herbicide include methyl 4-hydroxyphenylacetate.

Preferably, the herbicide may further include a herbicidal active ingredient having no antagonistic effect with the methyl 4-hydroxyphenylacetate.

Preferably, the methyl 4-hydroxyphenylacetate in the herbicide may have a content of 0.1-99%.

Preferably, a dosage form of the herbicide may include an aqueous solution.

The present disclosure further provides a method for preparing the above herbicide, including the following steps: pre-dissolving the methyl 4-hydroxyphenylacetate in an organic solvent, and then mixing with water to obtain a methyl 4-hydroxyphenylacetate herbicide aqueous solution.

Compared with the prior art, the present disclosure has the following beneficial effects:

It is found in the present disclosure that the herbicide using the methyl 4-hydroxyphenylacetate as an active ingredient has a contact killing effect on perennial herbs or Half-shrubby plants, especially the Eupatorium adenophorum. After spraying for 4 to 5 days, the Eupatorium adenophorum may be basically withered, which improves the control effect of the Eupatorium adenophorum and avoids the interference of topography on the control of the Eupatorium adenophorum.

The herbicide using the methyl 4-hydroxyphenylacetate as an active ingredient in the present disclosure has low toxicity, is almost non-toxic, and has no residue of other chemicals after use. Only a very small amount of the solvent may fall into the soil, which will not damage physical and chemical properties of the soil and the ecological environment, and has no teratogenic, mutagenic and carcinogenic effects on animals.

### BRIEF DESCRIPTION OF THE DRAWINGS

FIG. 1 shows a control effect of a herbicide aqueous solution in Example 1 on Eupatorium adenophorum, where A in FIG. 1 shows Eupatorium adenophorum before spraying, and B in FIG. 1 shows Eupatorium adenophorum after spraying for 5 days;

FIG. 2 shows a control effect of a herbicide aqueous solution in Example 3 on the Eupatorium adenophorum, where A in FIG. 2 shows Eupatorium adenophorum before spraying, and B in FIG. 2 and C in FIG. 2 show Eupatorium adenophorum after spraying for 5 days;

FIG. 3 shows a control effect of 50% ethanol on the Eupatorium adenophorum, where A in FIG. 3 shows Eupatorium adenophorum after spraying for 1 minute, and B in FIG. 3 shows Eupatorium adenophorum after spraying for 5 days; and

FIG. 4 shows a control effect of the herbicide aqueous solution in Example 1 on Nicotiana benthamiana, where A in FIG. 4 shows Nicotiana benthamiana before spraying, B in FIG. 4 shows Nicotiana benthamiana after spraying for 5 days, and C in FIG. 4 shows Nicotiana benthamiana after spraying for 8 days.

### DETAILED DESCRIPTION OF THE EMBODIMENTS

The present disclosure provides a method for using methyl 4-hydroxyphenylacetate in control of plants. The plants include perennial herbs or Half-shrubby plants, preferably Eupatorium adenophorum.

The methyl 4-hydroxyphenylacetate in the present disclosure can be a conventional commercially available product.

The methyl 4-hydroxyphenylacetate in the present disclosure has a use concentration greater than 150 mmol/L, preferably 200-300 mmol/L, more preferably 220-270 mmol/L, and most preferably 250 mmol/L. It is found in the present disclosure that when the methyl 4-hydroxyphenylacetate has a use concentration greater than 150 mmol/L, it has a contact killing effect on the Eupatorium adenophorum. Tender leaves and stems can be completely killed by spraying once, and the old leaves are basically withered after spraying for 4-5 days.

The present disclosure further provides a method for using methyl 4-hydroxyphenylacetate in herbicides. The herbicide can be used for controlling the Eupatorium adenophorum. As an optional implementation, the agricultural chemical can be used for controlling weeds such as the Eupatorium adenophorum in tobacco fields.

The present disclosure further provides a herbicide. Active ingredients of the herbicide include methyl 4-hydroxyphenylacetate. In the present disclosure, the methyl 4-hydroxyphenylacetate can exist as the only active ingredient of the herbicide, and may also be used in combination with other herbicidal active ingredients having no antagonistic effect with the methyl 4-hydroxyphenylacetate.

The herbicide of the present disclosure may be added with adjuvants for preparations such as surfactants, wetting agents, adhesives, thickeners, preservatives, colorants, and stabilizers as required.

In the present disclosure, the methyl 4-hydroxyphenylacetate in the herbicide has a content of 0.1-99%, preferably 10-80%.

The present disclosure further provides a method for preparing the above herbicide, including the following steps: pre-dissolving the methyl 4-hydroxyphenylacetate in an organic solvent, and then mixing with water to obtain a methyl 4-hydroxyphenylacetate herbicide aqueous solution.

The organic solvent of the present disclosure includes, but is not limited to, any one or a mixture of several miscible organic solvents such as ethanol, methanol, diethyl ether, dimethyl sulfoxide (DMSO), petroleum ether, and dichloromethane in any proportion. For the consideration of environmental protection, the organic solvent is preferably ethanol, more preferably an ethanol solution with a concentration of 20-50%.

In the present disclosure, the methyl 4-hydroxyphenylacetate is pre-dissolved in absolute ethanol, and then mixed with water to obtain a methyl 4-hydroxyphenylacetate herbicide aqueous solution. The methyl 4-hydroxyphenylacetate and the absolute ethanol have a mass volume ratio of 0.1-0.3 g/mL, preferably 0.15-0.25 g/mL, and more preferably 0.2 g/mL The addition amount of the water is 3-5 times the volume of the absolute ethanol. In the present disclosure, the methyl 4-hydroxyphenylacetate is rapidly dissolved in the absolute ethanol, and then water is added to reduce the concentration of the ethanol to obtain the herbicide. Preferably, the ethanol solution in the herbicide has a final concentration greater than 20%.

The technical solution provided by the present disclosure will be described in detail below with reference to examples, but they should not be considered as limiting the protection scope of the present disclosure.

Methyl 4-hydroxyphenylacetate in examples of the present disclosure (trade name: methyl 4-hydroxyphenylacetate and batch number: F20024)

### Example 1

The present example provided a methyl 4-hydroxyphenylacetate herbicide aqueous solution.

The methyl 4-hydroxyphenylacetate was purchased on the market. 2.07712 g of methyl 4-hydroxyphenylacetate was weighed, and added with 10 mL of absolute ethanol for dissolution. 40 mL of tap water was poured to obtain 50 mL of an aqueous solution with a concentration of 250 mmol/L.

### Example 2

The present example provided a methyl 4-hydroxyphenylacetate herbicide aqueous solution.

The methyl 4-hydroxyphenylacetate was purchased on the market. 2.32638 g of methyl 4-hydroxyphenylacetate was weighed, and added with 15 mL of absolute ethanol for dissolution. 35 mL of tap water was poured to obtain 50 mL of an aqueous solution with a concentration of 280 mmol/L.

### Example 3

The present example provided a methyl 4-hydroxyphenylacetate herbicide aqueous solution.

The methyl 4-hydroxyphenylacetate was purchased on the market. 1.329 g of methyl 4-hydroxyphenylacetate was weighed, and added with 10 mL of absolute ethanol for dissolution. 40 mL of tap water was poured to obtain 50 mL of an aqueous solution with a concentration of 160 mmol/L.

### Example 4

The present example provided a methyl 4-hydroxyphenylacetate herbicide aqueous solution.

The methyl 4-hydroxyphenylacetate was purchased on the market. 1.4956 g of methyl 4-hydroxyphenylacetate was weighed, and added with 10 mL of absolute ethanol for dissolution. 40 mL of tap water was poured to obtain 50 mL of an aqueous solution with a concentration of 180 mmol/L.

### Example 5

The present example provided a methyl 4-hydroxyphenylacetate herbicide aqueous solution.

The methyl 4-hydroxyphenylacetate was purchased on the market. 1.6617 g of methyl 4-hydroxyphenylacetate was weighed, and added with 10 mL of DMSO for dissolution. 40 mL of tap water was poured to obtain 50 mL of an aqueous solution with a concentration of 200 mmol/L.

### Comparative Example 1

The present example provided a methyl 4-hydroxyphenylacetate herbicide aqueous solution.

The methyl 4-hydroxyphenylacetate was purchased on the market. 1 g of methyl 4-hydroxyphenylacetate was weighed, and added with 10 mL of absolute ethanol for dissolution. 40 mL of tap water was poured to obtain 50 mL of an aqueous solution with a concentration of 120 mmol/L.

### Comparative Example 2

The present example provided a methyl 4-hydroxyphenylacetate herbicide aqueous solution.

The methyl 4-hydroxyphenylacetate was purchased on the market. 1.82787 g of methyl 4-hydroxyphenylacetate was weighed, and added with 50 mL of a 15% absolute ethanol solution for dissolution. It was found that the methyl 4-hydroxyphenylacetate could not be sufficiently dissolved, and 50 mL of white suspension was obtained.

### Test Example 1

(1) 25 mL of the methyl 4-hydroxyphenylacetate herbicide aqueous solution prepared in Example 1 was taken and sprayed evenly on the surface of Eupatorium adenophorum plants.

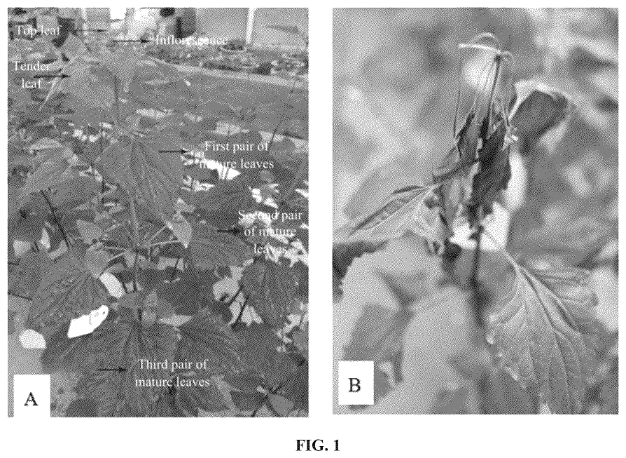

When not sprayed, the Eupatorium adenophorum plant had one pair of top leaves, one head inflorescence, one pair of tender leaves under the top leaves, and three pairs of mature leaves. The plant was healthy and vigorous, with a height of 40-50 cm. After spraying for 5 days, in the A plant, one pair of top leaves, one head inflorescence, one pair of tender leaves and stems below the top leaves were completely dead. The uppermost one pair of mature leaves among the three pairs of mature leaves withered in a large area. The lower two pairs of mature leaves began to wither from the edge inwards, and the axillary buds also died. These deaths were irreversible. The surrounding sprayed leaves also died at the same time, and the results are shown in FIG. 1 (A in FIG. 1 shows the plant before spraying, and B in FIG. 1 shows the plant after spraying for 5 days).

It can be seen from FIG. 1 that the methyl 4-hydroxyphenylacetate herbicide aqueous solution has an obvious contact killing effect on the Eupatorium adenophorum, which can cause irreversible death of the leaves, inflorescences, stems and axillary buds of the plant.

(2) 25 mL of the methyl 4-hydroxyphenylacetate herbicide aqueous solution prepared in Example 3 was taken and sprayed evenly on the surface of Eupatorium adenophorum plants.

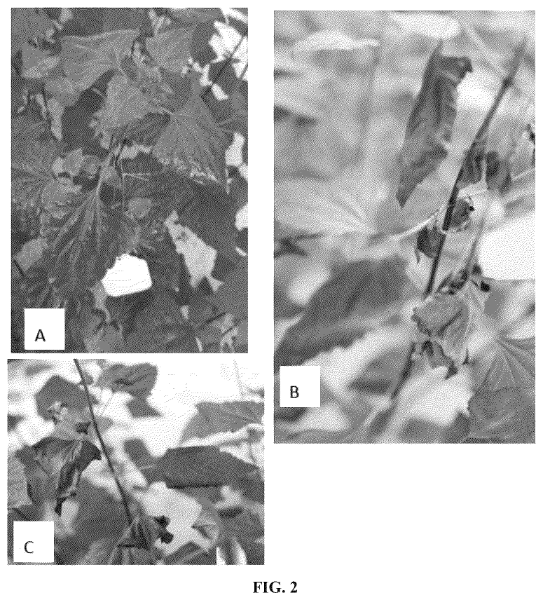

When not sprayed, the Eupatorium adenophorum plant had one pair of top leaves, one head inflorescence, one pair of tender leaves under the inflorescence, and four pairs of mature leaves. The plant was healthy and vigorous, with a height of 40-50 cm. After spraying for 5 days, one head inflorescence, one pair of tender leaves under the inflorescence, one pair of top leaves, and two pairs of mature leaves all died, and the lowermost two pairs of mature leaves had 50% yellow spots on the surface. The results are shown in FIG. 2 (A in FIG. 2 shows the plant before spraying, and B in FIG. 2 and C in FIG. 2 show the plant after spraying for 5 days).

(3) 25 mL of a 50% ethanol aqueous solution was directly taken and sprayed evenly on the surface of Eupatorium adenophorum plants.

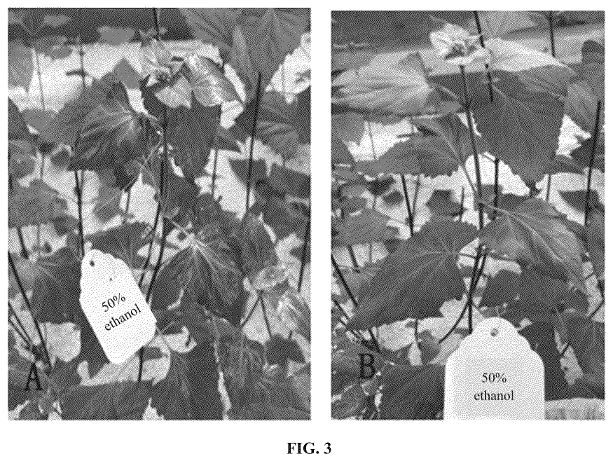

When not sprayed, the Eupatorium adenophorum plant had one pair of top leaves, one head inflorescence, one pair of tender leaves under the inflorescence, and four pairs of mature leaves. The plant was healthy and vigorous, with a height of 40-50 cm. After spraying for 5 days, the growth status of the Eupatorium adenophorum was not affected, and the results are shown in FIG. 3 (A in FIG. 3 shows the plant after spraying for 1 minute, and B in FIG. 3 shows the plant after spraying for 5 days).

It can be seen from FIG. 3 that only spraying 50% ethanol solution has no effect on the growth of the Eupatorium adenophorum, indicating that ethanol solutions with a concentration less than or equal to 50% will not affect the growth of the Eupatorium adenophorum. However, when the methyl 4-hydroxyphenylacetate is added to the ethanol solution with a concentration of 20% or 20-50%, the solution has a contact killing effect on the Eupatorium adenophorum. It is proved that the contact killing effect of the solutions obtained in Example 1 and Example 3 on the Eupatorium adenophorum is caused by the addition of the methyl 4-hydroxyphenylacetate.

(4) 25 mL of the methyl 4-hydroxyphenylacetate herbicide aqueous solution prepared in Example 1 was taken and sprayed evenly on the surface of Nicotiana benthamiana plants.

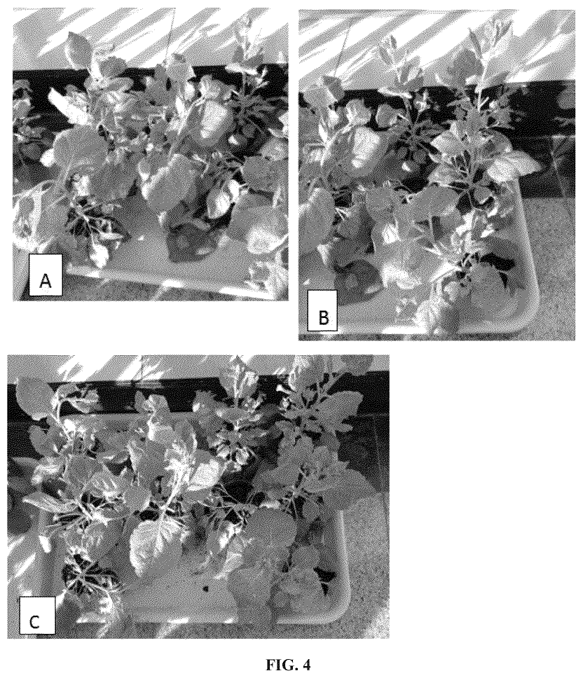

When not sprayed, the Nicotiana benthamiana plant was healthy and vigorous, with a height of 20-40 cm. After spraying for 5 days, the Nicotiana benthamiana grew normally without withering and death. After spraying for 8 days, the Nicotiana benthamiana did not die and grew normally. The results are shown in FIG. 4 (A in FIG. 4 shows the plant before spraying. B in FIG. 4 shows the plant after spraying for 5 days, and C in FIG. 4 shows the plant after spraying for 8 days.).

It can be seen from FIG. 4 that the methyl 4-hydroxyphenylacetate herbicide aqueous solution has no contact killing effect on the Nicotiana benthamiana, and can be used for controlling weeds such as the Eupatorium adenophorum in tobacco fields.

(5) 25 mL of the methyl 4-hydroxyphenylacetate herbicide aqueous solutions prepared in Comparative Examples 1 to 2 were taken and sprayed evenly on the surface of Eupatorium adenophorum plants. It was found that it had little effect on the growth of the Eupatorium adenophorum, and there was no stem and leaf withering.

The results shows that if the methyl 4-hydroxyphenylacetate has a too low concentration, or the organic solvent has a too low concentration to effectively dissolve the methyl 4-hydroxyphenylacetate, the contact killing effect of the obtained solution on the Eupatorium adenophorum will be affected.

The above descriptions are merely preferred implementations of the present disclosure. It should be noted that those of ordinary skill in the art may further make several improvements and modifications without departing from the principle of the present disclosure, but such improvements and modifications should be deemed as falling within the protection scope of the present disclosure.

What is claimed is:

1. Method for controlling a plant, wherein comprising using methyl 4-hydroxyphenylacetate.

2. The method according to claim 1, wherein the plants comprise a perennial herb or Half-shrubby plant.

3. The method according to claim 2, wherein the plant is Eupatorium adenophorum.

4. The method according to any one of claim 1, wherein the methyl 4-hydroxyphenylacetate has a use concentration greater than to 150 mmol/L.

5. Use of methyl 4-hydroxyphenylacetate in herbicides.

6. A herbicide, wherein active ingredients of the herbicide comprise methyl 4-hydroxyphenylacetate.

7. The herbicide according to claim 6, further comprising a herbicidal active ingredient having no antagonistic effect with the methyl 4-hydroxyphenylacetate.

8. The herbicide according to claim 6, wherein the methyl 4-hydroxyphenylacetate in the herbicide has a content of 0.1-99%.

9. The herbicide according to claim 6, wherein a dosage form of the herbicide comprises an aqueous solution.

10. A method for preparing the herbicide according to claim 6, comprising: pre-dissolving the methyl 4-hydroxyphenylacetate in an organic solvent, and mixing with water to obtain a methyl 4-hydroxyphenylacetate herbicide aqueous solution.

11. The method according to 4, wherein the plants comprise a perennial herb or Half-shrubby plant.

12. The method according to 5, wherein the plant is Eupatorium adenophorum.

13. The herbicide according to claim 9, further comprising a herbicidal active ingredient having no antagonistic effect with the methyl 4-hydroxyphenylacetate.

14. The herbicide according to claim 9, wherein the methyl 4-hydroxyphenylacetate in the herbicide has a content of 0.1-99%.

15. The method according to claim 10, further comprising a herbicidal active ingredient having no antagonistic effect with the methyl 4-hydroxyphenylacetate.

16. The method according to claim 10, wherein the methyl 4-hydroxyphenylacetate in the herbicide has a content of 0.1-99%.

17. The method according to claim 10, wherein a dosage form of the herbicide comprises an aqueous solution.
